(19) United States
(12) Patent Application Publication
(10) Pub. No.: US 2014/0048977 A1
(43) Pub. Date: Feb. 20, 2014

(54) MOLDING OF A NEUROSTIMULATOR FOR DELIVERY INTO THE PTERYGOPALATINE FOSSA

(71) Applicant: AUTONOMIC TECHNOLOGIES, INC., Redwood City, CA (US)

(72) Inventors: Ryan Powell, Sunnyvale, CA (US); Carl Lance Boling, San Jose, CA (US); Jennifer Teng, San Francisco, CA (US); Morgan Clyburn, Menlo Park, CA (US)

(73) Assignee: AUTONOMIC TECHNOLOGIES, INC., Redwood City, CA (US)

(21) Appl. No.: 13/966,402

(22) Filed: Aug. 14, 2013

Related U.S. Application Data
(60) Provisional application No. 61/684,230, filed on Aug. 17, 2012.

(51) Int. Cl. A61N 1/36

(52) U.S. Cl. CPC A61N 1/36075
USPC 264/279; 425/117

(57) ABSTRACT

A method and apparatus for molding a medical device utilizes a rigid outer stiffener and a flexible inner mold that nests with the outer stiffener. The medical device can be a stimulating apparatus used to deliver electrical stimulation to a peripheral, central or autonomic neural structure. More specifically, the medical device can be a neurostimulator apparatus designed to delivery electrical stimulation to the sphenopalatine ganglion (SPG) to treat primary headaches, such as migraines, cluster headaches and/or many other neurological disorders, such as atypical facial pain and/or trigeminal neuralgias.

### RELATED APPLICATIONS

This application claims the benefit of U.S. Provisional Application Ser. No. 61/684,230, filed on Aug. 17, 2012, the disclosure of which is hereby incorporated by reference in its entirety.

### TECHNICAL FIELD

The invention relates generally to a stimulating apparatus used to deliver electrical stimulation to a peripheral, central, or autonomic neural structure. More particularly, the invention relates to a molded medical device and a method of manufacturing the molded medical device. More specifically, the medical device can be a neurostimulator apparatus designed to delivery electrical stimulation to the sphenopalatine ganglion (SPG) to treat primary headaches, such as migraines, cluster headaches and/or many other neurological disorders, such as atypical facial pain and/or trigeminal neuralgias.

### BACKGROUND OF THE INVENTION

Primary headaches are debilitating ailments that afflict millions of individuals worldwide. The specific pathophysiology of primary headaches is not known. Known causes of headache pain include trauma, vascular defects, autoimmune deficiencies, degenerative conditions, infections, drug and medication-induced causes, inflammation, neoplastic conditions, metabolic-endocrine conditions, iatrogenic conditions, musculoskeletal conditions, and myofacial causes. In many situations, however, even though the underlying cause of the headache may be identified and treated, the headache pain itself may persist.

Recent clinical studies in treatment of headaches have targeted the manipulation of sphenopalatine (pterygopalatine) ganglion (SPG), a large, extra-cranial parasympathetic ganglion. A ganglion is a mass of neural tissue found in some peripheral and autonomic nerves. Ganglia are located on the roots of the spinal nerves and on the roots of the trigeminal nerve. Ganglia are also located on the facial, glossopharyngeal, vagus and vestibulochoclear nerves. The SPG is a complex neural ganglion with multiple connections, including autonomic, sensory, and motor connections. The SPG includes parasympathetic neurons that innervate, in part, the middle cerebral and anterior cerebral blood vessels, the facial blood vessels, and the lacrimal glands.

The maxillary branch of the trigeminal nerve and the nerve of the pterygoid canal (also known as the vidian nerve which is formed by the greater and deep petrosal nerves) send neural projections to the SPG. The fine branches from the maxillary nerve (pterygopalatine nerves) form the sensory component of the SPG. These nerve fibers pass through the SPG and do not synapse. The greater petrosal nerve carries the preganglionic parasympathetic axons from the superior salivary nucleus, located in the pons, to the SPG. These fibers synapse onto the postganglionic neurons within the SPG. The deep petrosal nerve connects the superior cervical sympathetic ganglion to the SPG and carries postganglionic sympathetic axons that again pass through the SPG without any synapsing in the SPG.

The SPG is located within the pterygopalatine fossa (PPF). The PPF is bounded anteriorly by the maxilla, posteriorly by the medial plate of the pterygoid process and greater wing of the sphenoid process, medially by the palatine bone, and superiorly by the body of the sphenoid process. The lateral border of the PPF is the pterygomaxillary fissure, which opens to the infratemporal fossa.

Various clinical approaches have been used to modulate the function of the SPG in order to treat headaches, such as cluster headaches or chronic migraines. These approaches vary from lesser or minimally invasive procedures (e.g., transnasal anesthetic blocks) to procedures or greater invasiveness (e.g., surgical ganglionectomy). Other procedures of varying invasiveness include those such as surgical anesthetic injections, ablations, gamma knife procedures, and cryogenic surgery. Although most of these procedures can exhibit some short term efficacy in the order of days to months, the results are usually temporary and the headache pain eventually reoccurs.

### SUMMARY OF THE INVENTION

According to one aspect of the invention, an apparatus is configured to mold a medical device. The apparatus includes a rigid, two-piece, outer stiffener and a flexible, two-piece, mold, which together, will allow for the over-molding of a medical device, specifically a neurostimulator apparatus designed to delivery electrical stimulation to the sphenopalatine ganglion (SPG).

According to another aspect of the invention, a method is provided for molding a medical device, specifically a neurostimulator apparatus designed to deliver electrical stimulation to the sphenopalatine ganglion (SPG). The method includes the step of creating a rigid, two-piece outer stiffener that retains and provides structural support for a flexible, two-piece inner mold. This inner mold provides the geometry for the over-molding of the medical device. A pre-over-molded medical device is placed in the flexible inner mold which is then supported by the outer stiffener. A biocompatible reaction-injection-molding (RIM) polymer is injected into the inner mold to over-mold the medical device. The flexible inner mold is then removed from the rigid outer stiffener. The medical device can then be extracted by peeling away the flexible inner mold.

### BRIEF DESCRIPTION OF THE DRAWINGS

The foregoing and other features of the invention will become apparent to those skilled in the art to which the invention relates upon reading the following description with reference to the accompanying drawings, in which:

FIG. 1 illustrates an implantable medical device implanted in a patient;

FIG. 2 is a schematic perspective illustration of an apparatus for manufacturing a medical device, such as the medical device of FIG. 1;

FIG. 3 is a diagram illustrating a method for manufacturing a medical device, such as the medical device of FIG. 1.

### DESCRIPTION

The invention relates generally to a stimulating apparatus used to deliver electrical stimulation to a peripheral, central or autonomic neural structure. More specifically, the invention relates to molding a medical device, specifically a neurostimulator apparatus designed to delivery electrical stimulation to the sphenopalatine ganglion (SPG) to treat primary headaches, such as migraines, cluster headaches and/or many other neurological disorders, such as atypical facial pain and/or trigeminal neuralgias.

According to one aspect of the invention, an apparatus for manufacturing a medical device includes a rigid, two-piece, outer stiffener and a flexible, two-piece, inner mold. Together, the outer mold and inner mold are used to over-mold the medical device, specifically a neurostimulator apparatus designed to delivery electrical stimulation to the sphenopalatine ganglion (SPG). The neurostimulator can be employed to assist in treating a variety of chronic or acute medical conditions. Examples of such medical conditions can include, but are not limited to, pain (e.g., headache and/or facial pain), movement disorders, epilepsy, cerebrovascular diseases, autoimmune diseases, sleep disorders, autonomic disorders, neurological disorders, urinary bladder disorders, abnormal metabolic states, disorders of the muscular system, and neuropsychiatric disorders.

A brief discussion of the pertinent anatomy and neurophysiology is provided to assist the reader in understanding the subject invention. The autonomic nervous system innervates numerous pathways within the human body and consists of two divisions: the sympathetic and the parasympathetic nervous systems. The sympathetic and parasympathetic nervous systems are antagonistic in their action, balancing the other system's effects within the body. The sympathetic nervous system (SNS) usually initiates activity within the body, preparing the body for action, while the parasympathetic nervous system (PNS) primarily counteracts the effects of the SNS.

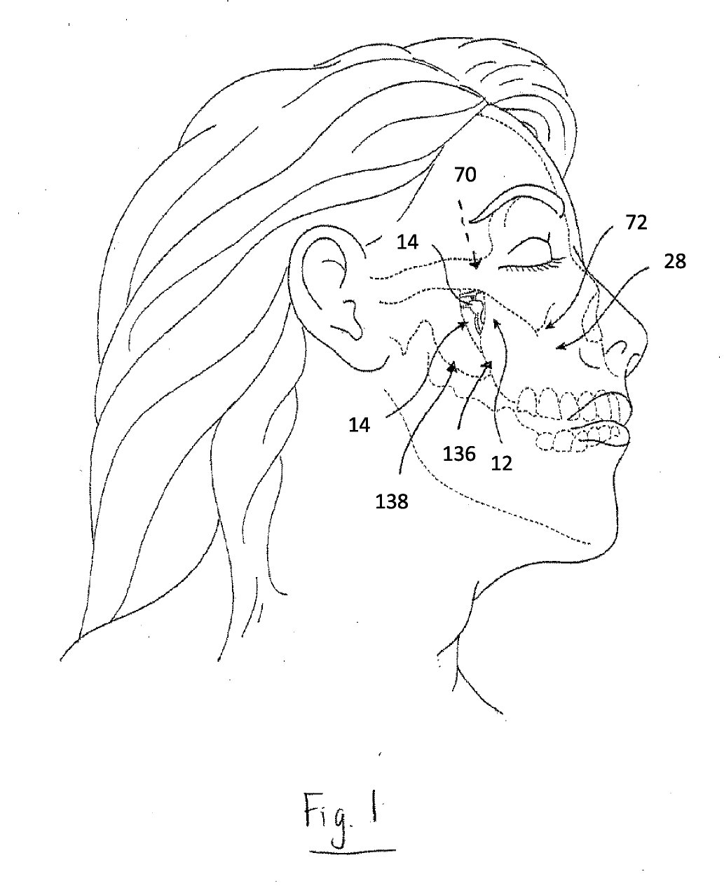

Referring to FIG. 1, the sphenopalatine ganglia (SPG) 10 are located on both sides of the head. For purposes of this description of the invention, reference is made to the SPG 10 located on the left side of the head. Those skilled in the art will appreciate that the invention could also be described in conjunction with the SPG on the right side of the head. As shown in FIG. 1, the SPG 10 is located behind the posterior maxilla 12 in the PPF 14, posterior to the middle nasal turbinate (not shown in detail). The SPG 10 is part of the parasympathetic division of the autonomic nervous system; however, the SPG has both sympathetic and parasympathetic nerve fibers, as well as sensory and motor nerve fibers either synapsing within the ganglion (e.g., parasympathetic) or fibers that are passing through the ganglion and not synapsing (e.g., sympathetic, sensory and motor).

The parasympathetic activity of the SPG 10 is mediated through the greater petrosal nerve (not shown), while the sympathetic activity of the SPG is mediated through the deep petrosal nerve (not shown), which is essentially an extension of the cervical sympathetic chain (not shown). Sensory sensations generated by or transmitted through the SPG 10 include, but are not limited to, sensations to the upper teeth, feelings of foreign bodies in the throat, and persistent itching of the ear. The SPG 10 transmits sensory information, including pain, to the trigeminal system via the maxillary division and ophthalmic division (not shown).

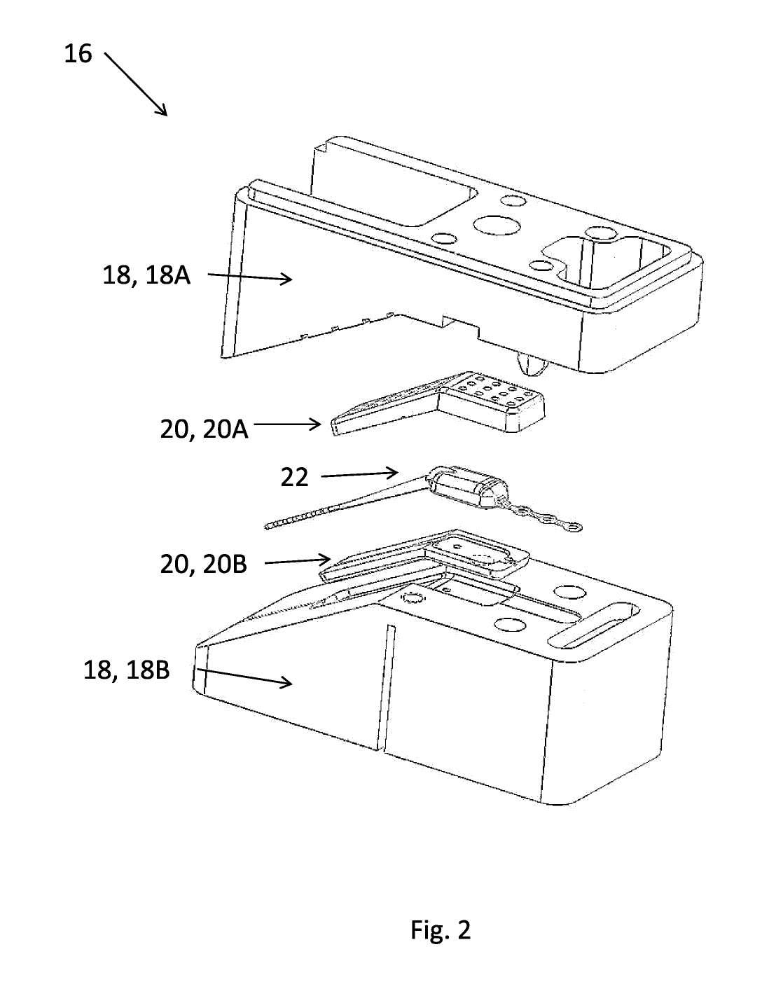

Referring to FIG. 2, according to one aspect of the invention, an apparatus 16, in the form of a tool is configured to mold a neurostimulator for delivery into a craniofacial region of a subject. The tool 16 comprises a rigid, two-piece outer stiffener 18 including first (upper) and second (lower) stiffener parts 18a and 18b, respectively. The tool 16 also includes a flexible, two-piece inner mold 20 including first (upper) and second (lower) inner mold parts 20a and 20b, respectively.

Together, the outer stiffener 18 and inner mold 20 of the tool 16 can be used to manufacture a medical device. More particularly, the tool 16 can be used to form a molded portion of a medical device. For example, the tool 16 can be used to manufacture or form a molded portion of a neurostimulator 22, such as that disclosed in U.S. patent application Ser. No. 12/765,712 (hereinafter, “the '712 application”), the disclosure of which is hereby incorporated by reference in its entirety.

The neurostimulator 20 can have a variety of configurations. As such the neurostimulator 22 can generally include any active implantable medical device configured to deliver electrical stimulation, alone or in combination with other types of stimulation to tissue of a subject. The neurostimulator 22 can also include any active implantable medical device configured for implantation for a relatively short period of time (e.g., to address acute medical conditions) or a relatively long period of time (e.g., to address chronic medical conditions). Additionally, the neurostimulator 22 can include one or more elements used to record or monitor a physiological response of a subject's tissue (e.g., a delivered therapy), as well as one or more other components that interface with the patient's tissue (e.g., therapeutic agent delivery mechanisms, sensors, etc.).

According to one example, the neurostimulator 22 can be implanted as disclosed in the '712 application, i.e., such that the stimulator body is positioned sub-periosteally medial to the zygoma 70 (FIG. 1) on the posterior maxilla 12 within the buccal fat pad (not shown) of the cheek, and the integral fixation apparatus is anchored to the zygomaticomaxillary buttress 72 (FIG. 1) such that the integral stimulation lead is placed within the PPF 14 (FIG. 1) or, more specifically, in close proximity (e.g., about 1-5 mm) to the SPG 10.

According to another aspect of the invention, the first and second stiffener portions 18a and 18b of the outer stiffener 18 comprise top and bottom portions, respectively, that fit together snuggly when closed. By “snuggly,” it is meant that the first and second stiffner portions 18a and 18b are constructed with close tolerances selected such that the portions have a tight, secure fit suitable for facilitating and withstanding the conditions (e.g., temperatures, pressures, stresses, forces) associated with RIM molding processes. The first (upper) stiffener portion 18a and second (lower) stiffener portion 18b can be made of a rigid or semi-rigid metal or metal alloy, such as stainless steel or aluminum, or a hard plastic, such as PTFE blocks or epoxy resins and the like. Additionally, due to the durability of the construction materials, the first and second stiffener portions 18a and 18b can be used repetitively to over-mold multiple parts.

According to another aspect of the invention, the first and second inner mold portions 20a and 20b of the inner mold 20 comprise top and bottom portions, respectively. The first (upper) inner mold portion 20a and second (lower) inner mold portion 20b nest within the first and second outer stiffener portions 18a and 18b, respectively, and fit together snuggly with the stiffener portions and with each other when closed. Again, by “snuggly,” it is meant that the first and second inner mold portions 20a and 20b are constructed with close tolerances selected such that the portions have a tight, secure fit suitable for facilitating and withstanding the conditions (e.g., temperatures, pressures, stresses, forces) associated with RIM molding processes. The first and second inner mold portions 20a and 20b can be made of a highly flexible material such as an elastomer or the like.

The flexible elastomeric inner mold 20 is received in the stiffener 18 and nests and mates with the form of the recess or mold cavity in which it is received. Since the stiffener 18 is constructed such that the mold cavity matches the outer shape, dimensions, and contour of the inner mold 20, the inner mold is received with a close tolerance fit. Due to this fit, the stiffener 18 maintains the shape and configuration of the inner mold 20, adding to the structural integrity, i.e., the stiffness of the inner mold. Because the fit is close, the inner mold 20 can resist distortion in response to receiving the injected polymer due to the stiffness leant to the inner mold by the stiffener 18.

Those skilled in the art will appreciate that conventional molding techniques for producing a medical device can have certain drawbacks. For example, conventional molds constructed of a rigid, durable, e.g., metal, material can exhibit particular problems with wear and damage. For example, the conventional molds wear easily, which can lead to excessive wear over time due to the removal of finished parts. Additionally, the removal of parts directly from the rigid molds risks damage to both the finished part and the mold itself each time this task is performed. Additionally, damage to the mold compromises the quality of the molding on subsequent parts. Furthermore, those skilled in the art will appreciate that rigid molds may not allow for molding parts whose design include portions with a negative draft. All of these risks are particularly undesirable since the conventional mold parts are expensive to produce and replace.

Advantageously, according to the invention, the construction of the tool 16, including in combination the rigid outer stiffener 18 and flexible inner mold 20, facilitates improved manufacture of the medical device, e.g., neurostimulator 22. The rigid construction of the outer stiffener 18 provides a level of durability and reusability to the tool 16, whereas the flexible construction of the inner mold 20 allows for ease in extracting the finished part from the tool 16 while minimizing the risk of damage to the to the finished part. Additionally, the flexible inner mold also allows for medical device designs that include portions with negative draft because the flexible mold can simply peel away from the negative draft portions. The inner mold 20 can be constructed to have a limited number of usages, including a single use, in which case the inner mold 20 would be a disposable, single use part.

From the above, those skilled in the art will appreciate that the tool 16 is used to over-mold or insert mold a medical device, such as the neurostimulator 22. To assemble the tool 16, the inner mold portions 20a and 20b are placed within the stiffener portions 18a and 18b. The medical device 22 is placed in the inner mold 20 portion and the tool 16 is closed. Then, un-cured liquid polymer over-mold material (e.g., a biocompatible polymer such as a reaction injection molding (RIM) polymer) is injected into the tool 16 and over-molds the medical device. During molding, the stiffener 18 supports the inner mold 20, which allows the inner mold to maintain the prescribed form of the injected polymer material during the molding process.

After the polymer is cured, the tool 16 is opened and the inner mold 20 is removed with the over-molded medical device 22 encased therein. Removal of the relatively soft, flexible inner mold 20 allows for the extraction of the medical device 22 with minimal risk of damage to the device or to the tool 16, especially the stiffener 18. Damaging the inner mold 20 is not a concern because it is disposable. A new inner mold 20 can then be placed in the stiffener portion 18 and the process repeated to produce subsequent medical devices 22.

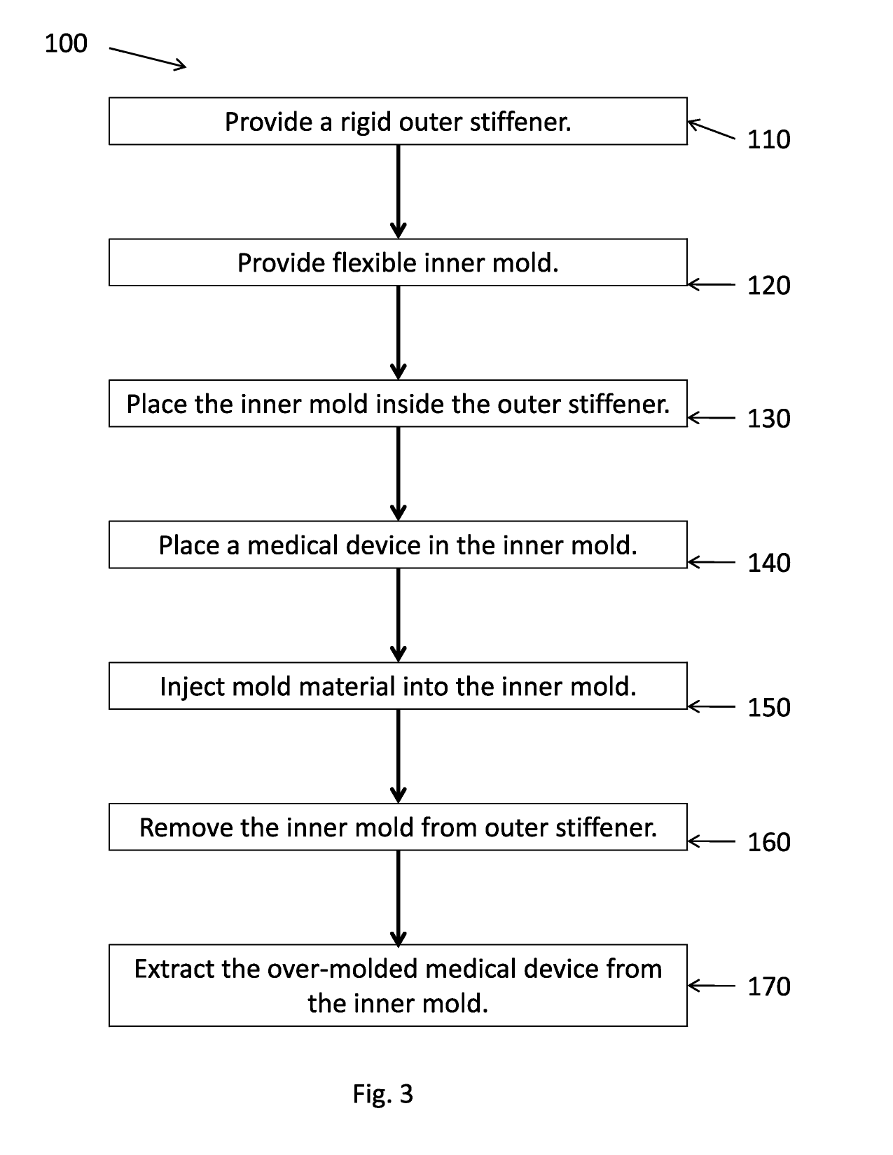

According to another aspect of the invention, FIG. 3 illustrates a method 100 for producing a medical device, such as the neurostimulator 22 designed to deliver electrical stimulation to the sphenopalatine ganglion (SPG). The method 100 includes the step 110 of providing a rigid outer stiffener, such as the rigid two-piece outer stiffener 20 illustrated in FIG. 2. The method 100 also includes the step 120 of providing a flexible inner mold, such as the flexible two-piece inner mold 20 illustrated in FIG. 2.

The method 100 also includes the step 130, of placing the inner mold inside the outer stiffener. The method 100 also includes the step 140 of placing a medical device is placed inside the inner mold. The method 100 also includes the step 150 of injecting a mold material into the inner mold to over-mold the medical device. The mold material can be a biocompatible RIM polymer.

The method 100 also includes the step 160 or removing the inner mold from the outer stiffener. The method 100 includes the further step 170 of extracting the over-molded medical device flexible inner mold.

Accordingly, those skilled in the art will appreciate that the invention provides an apparatus 16 and method 100 for manufacturing an over-molded medical device. The apparatus 16 and method 100 are advantageous in that the risk of damage to both the apparatus and to the manufactured medical device, and the costs associated therewith, are minimized.

From the above description of the invention, those skilled in the art will perceive improvements, changes and modifications. Such improvements, changes and modifications within the skill of the art are intended to be covered by the appended claims.

1. An apparatus for over-molding a medical device, the apparatus comprising:
a rigid outer stiffener; and
a flexible inner mold that nests with the outer stiffener.

2. The apparatus recited in claim 1, wherein the outer stiffener comprises first and second stiffener portions that fit together snuggly when closed.

3. The apparatus recited in claim 1, wherein the outer stiffener has a rigid metal construction.

4. The apparatus recited in claim 1, wherein the outer stiffener has a rigid hard plastic construction.

5. The apparatus recited in claim 1, wherein the outer stiffener is reusable and the inner mold is single use disposable.

6. The apparatus recited in claim 1, wherein the inner mold comprises first and second portions that which fit together snuggly when closed.

7. The apparatus recited in claim 1, wherein the inner mold is constructed of a flexible elastomer material that can be peeled away from the over-molded device.

8. The apparatus recited in claim 1, wherein the inner mold has a flexible construction that allows for extracting the inner mold and the molded medical device from the outer stiffener after molding.

9. The apparatus recited in claim 1, wherein the outer stiffener supports the flexible inner mold and helps maintain the form of the inner mold when the mold is in a closed condition.

10. A method for molding a medical device, the method comprising the steps of:
providing a rigid outer stiffener;
providing a flexible inner mold;
placing the inner mold inside the outer stiffener;
placing a medical device inside the inner mold; and
injecting a mold material into the inner mold.

11. The method recited in claim 10 further comprising the steps of:
removing the inner mold from the outer stiffener; and
extracting the over-molded medical device from the inner mold.
